(19) United States
(12) Patent Application Publication
(10) Pub. No.: US 2005/0244439 A1
(43) Pub. Date: Nov. 3, 2005

(54) Composition comprising anionic clay layered host material with intercalated functional-active organic compound

(75) Inventor: Joseph F. Bringley, Rochester, NY (US)

Correspondence Address: Paul A. Leipold;Patent Legal Staff, Eastman Kodak Company, 343 State Street, Rochester, NY, 14650-2201, US

(73) Assignee: Eastman Kodak Company

(21) Appl. No.: 10/837,189

(22) Filed: Apr. 30, 2004

(51) Int. Cl. A61K 9/00; A61K 9/14

(52) U.S. Cl. 424/400

(57) ABSTRACT

A composition comprising particles of an anionic clay layered host material and functional-active organic compound dispersed in an aqueous medium is described, wherein at least 80% of the finctional-active organic compound in the dispersion is intercalated between layers of the layered host material particles, and the stoichiometric ratio of anionic clay to functional-active compound is between about 1.2 to 7 equivalents. A process for the preparation of a composition comprising particles of an anionic clay layered host material and functional-active organic compound dispersed in an aqueous medium is also described, the process comprising reacting between about 1.2 to 7 equivalents of a calcined product of a layered double hydroxide with a functional-active organic compound in an aqueous medium, wherein a secondary non-functional-active ion is also reacted with the calcined product, such that the calcined product is rehydrated in the presence of the functional-active organic compound and secondary ions to form particles of an anionic clay layered double hydroxide host material with molecules of the functional-active organic compound and secondary ion intercalated between layers of the layered host material particles.

### FIELD OF THE INVENTION

The present invention relates to anionic clay layered host materials with intercalated functional-active organic compounds, and to a process for making aqueous dispersions of such compositions.

### BACKGROUND OF THE INVENTION

The sequestration and controlled release of components in a chemical system can dramatically impact the performance and efficacy of a chemical system. For example, the controlled release of molecular components in photographic systems has long been used to improve the efficiency of photographic films. More recently, the sequestration and controlled release of health and cosmetically important molecules, such as fragrances, has been employed to improve the performance of consumer products. Sequestration refers to the ability of a complex system to first sequester or “hide” a specific component of the system, from a desired target component, system, or user. In the sequestrated form, the component may not interact, or react with the target system or component. The sequestered component may then later be released, either slowly through simple diffusion or by means of a chemical or physical switch applied to the system, to perform a desired function.

The most well known examples of sequestration and controlled release of components are in controlled drug-delivery. Controlled drug-delivery is important for treating disease and sickness in human and other plant and animal species. Much effort has been applied to the design and development of systems able to deliver a specific dose of a drug slowly over time to improve the efficacy of drugs and to decrease the risk of side effects to the patient. Primarily, the effort to date has been focused on encapsulating drugs within polymers and allowing for the drug to slowly diffuse through the polymeric layer into the patient. This has the problem in that the methods of producing such materials are expensive and often the release of the drug cannot be controlled, but rather relies on simple permeation and diffusion processes.

More recently, Choy et al. in U.S. Pat. No. 6,329,525 B1 have described a bio-inorganic composite for retaining and carrying bio-materials with stability and reversible dissociativity, and methods for preparing the bio-inorganic composite. The bio-inorganic hybrids are based upon intercalation of bio-molecules into layered host materials known as layered double hydroxides (often called anionic clays). The bio-molecules are adsorbed by the layered host material and ensconced therein, but later may be released upon exposure to a change in acidity or a change in electrolyte concentration. The bio-inorganic hybrids thus serve as a potentially effective means to effect controlled delivery of bio-materials such as drugs, health-care reagents and biomaterials.

WO patent application 02/47729 A2 to O'Hare describes a drug delivery system comprising an intercalate of a layered double hydroxide having, before intercalation, layers of metal hydroxides, and having intercalated therein a pharmaceutically-active compound having at least one anionic group. The intercalated compositions may be employed to release a variety of drugs within the stomach of a patient ingesting the “drug delivery system”. The patent application also describes a method of preparing a layered double hydroxide having an intercalated pharmaceutically-active compound, which comprises treating an aqueous solution of the pharmaceutically-active compound with a layered double hydroxide where the pharmaceutically-active compound is present in a molar excess with respect to the layered double hydroxide, and separating the intercalate of the layered double hydroxide. The separation procedure generally involves filtration and washing of the product intercalate.

U.S. 2003/0129243 A1 to Pitard describes compositions comprising at least a nucleic acid and a mineral particle having an interchangeable foliate structure. The mineral particles employed are anionic clays (e.g., hydrotalcite) and the patent demonstrates aqueous compositions wherein, about 80-90% of the active chemistry (DNA :in the example of FIG. 5) is sequestered or removed from solution by the hydrotalcite, if the ratio of mineral (hydrotalcite) to DNA is greater than about 50:1 (w/w).

There is a problem in the prior art in that the described compositions are expensive to prepare, and typically require filtration and washing of the product. Filtration and washing can lead to loss and waste of the chemically-active, or pharmaceutically-active component, and diminishes the economic efficiency of the process. There is another problem in that the compositions typically require a very large amount of anionic clay to effectively sequester all the active material from solution. There is a further problem in that the compositions may release the active ingredient immediately upon exposure to an aqueous environment. There are still further problems in that the active components may be released prematurely or too quickly in the stomach of a patient and that the rate of release may be too high.

### SUMMARY OF THE INVENTION

In accordance with one embodiment, the invention is directed towards a composition comprising particles of an anionic clay layered host material and functional-active organic compound dispersed in an aqueous medium, wherein at least 80% of the functional-active organic compound in the dispersion is intercalated between layers of the layered host material particles, and the stoichiometric ratio of anionic clay to functional-active compound is between about 1.2 to 7 equivalents.

In accordance with a further embodiment, the invention is directed towards a process for the preparation of a composition comprising particles of an anionic clay layered host material and functional-active organic compound dispersed in an aqueous medium, the process comprising reacting between about 1.2 to 7 equivalents of a calcined product of a layered double hydroxide with a functional-active organic compound in an aqueous medium, wherein a secondary non-functional-active ion is also reacted with the calcined product, such that the calcined product is rehydrated in the presence of the functional-active organic compound and secondary ions to form particles of an anionic clay layered double hydroxide host material with molecules of the functional-active organic compound and secondary ion intercalated between layers of the layered host material particles.

### DETAILED DESCRIPTION OF THE INVENTION

In various embodiments, the invention relates to a composition for a chemical-delivery, and/or, a drug-delivery system, comprising an aqueous dispersion of an anionic clay, an intercalated functional-active organic compound and optionally a polymer, wherein at least 80% of the functional-active compound in the composition resides between the layers of the layered host material. The term “functional-active” is used herein to refer to, e.g., chemically-active, pharmaceutically-active, or nutraceutically-active ions, molecules, complexes or polymers. The invention further relates to a process for manufacturing a composition for a chemical-delivery, and/or, a drug delivery system comprising an aqueous dispersion comprising an anionic clay, an intercalated functional-active and optionally a polymer, which due to increased percentages of the functional-active compound being intercalated between the layers of the layered host material, may not require filtration or washing. The compositions of the invention are relatively simple and inexpensive to manufacture, and result in less loss or waste of the functional-active component. As substantially all of the functional-active organic compound may be intercalated in compositions in accordance with the invention, the invention provides a chemical-delivery and/or a drug-delivery system with significantly improved control over the rate of release of the functional-active component, especially for delivery of functional-active compounds in the stomach of a patient, and in a particular embodiment, control over the intravenous release of the intercalated functional-active component.

The chemical sequester and release concepts of the invention are based upon intercalation chemistry. Intercalation is a process in which a layered material, referred to as the host, swells or opens to accommodate other molecules or ions, referred to as the guest.
Host+guest⇄Host(guest)ₓ
Intercalation is, by definition, a reversible process and the guest molecule may diffuse from, or de-intercalate, from the interlayer space and are unaltered from the intercalation process. Layered compounds capable of sequestering ions and molecules by intercalation have been described in a number of publications. The choice of host material is dependent upon the particular molecule to be intercalated. A layered host material may be chosen which intercalates only cations, or conversely, only anions, or neutral molecules. The following publications are included for reference on this matter: “Intercalation Chemistry”, A. J. Jacobson and S. Whittingham, eds., Academic Press, NY 1982; “Intercalated Layered Materials”, F. Levy, D. Riedel Press, Dordrecht, Holland (1979); W. T. Reichle, CHEMTECH,, 16, 58 (1986); “An Introduction to Clay Colloid Chemistry”, H. van Olphen, 2^{nd} Ed., Krieger Pub. Co., Malabar, Fla. (1991).

The invention provides a chemical-delivery, and/or, a drug-delivery system comprising an anionic clay. Anionic clays are layered materials that are able to reversibly exchange (or intercalate) anions between their positively charged-layers. Anionic clays have been discussed by a number of authors. See, e.g., W. T. Reichle, “Synthesis of anionic clay minerals (mixed metal hydroxides, hydrotalcite) Solid State Ionics, 22, 135-141 (1986); F. Cavani, F. Trifiro and A. Vaccari, “Hydrotalcite-type anionic clays: preparation, properties and applications”, Catalysis Today, 11, 173-301 (1991); F. Trifiro and A. Vaccari, “Hydrotalcite-like anionic clays (Layered double hydroxides), in Comprehensive Supramolecular Chemistry, “Solid State Supramolecular Chemistry: Two- and Three-dimensional Inorganic Networks”, Alberti G.; Bein, T. Eds., Elsevier, New York, Chapter 8 (1996).

A specific example of anionic clays which may be employed in the invention are layered double hydroxides. Layered double hydroxides are closely related to the mineral “hydrotalcite”, and due to their unique ability to bind anions are conventionally referred to as anionic clays. Layered double hydroxides have the general formulas:
[M²⁺₁₋ₓM³⁺ₓ(OH)₂]^{x+}(x/n)Aⁿ⁻.yH₂O (I)
or
[M¹⁺M³⁺₂(OH)₆]¹⁺(1/n)Aⁿ⁻.yH₂O ; (II)
where M¹⁺ is a mono-valent metal selected from but not limited to Li, Na, K, Rb or Cs; M²⁺ is a divalent metal selected from but not limited to Ca, Mg, Mn, Co, Ni, Cu, Zn, and Cd; and M³⁺ is a trivalent metal selected from but not limited to Cr, Fe, Al, Ga, In, Mo; A is an anion chosen from OH⁻, NO₃⁻, F⁻, Cl⁻, Br⁻, I⁻, ClO₄⁻, SO₄²⁻, CO₃²⁻ or any other inorganic or organic anion, especially carboxylates and sulfonates, chosen such that the rule of charge neutrality is obeyed; n is an integer; x may be any rational number between 0 and 1 (typically between 0.01 an 0.5, more typically between 0.01 and 0.4); and y may be any rational number between 0 and 10. Anionic clays of the type described by formula (I) include Mg₂Al(OH)₆.1/2CO₃.yH₂O (note that this is equivalent to [Mg₁₋ₓAlₓ(OH)₂]0.165 CO₃.yH₂O for x=0.33); Zn₂Al(OH)₆.1/2CO₃.yH₂O; Mg₂Al(OH)₆.NO₃.yH₂O; and Zn₂Al(OH)₆.NO₃.yH₂O. Anionic clays of the type described by formula (II) include LiAl₂(OH)₆.Cl.yH₂O and NaAl₂(OH)₆.Cl.yH₂O.

The intercalation chemistry of anionic clays may proceed by one of three general mechanisms. The three intercalation reactions are (i) ion-exchange, (ii) direct precipitation and (iii) reconstruction synthesis. For purposes of illustration, the three types of intercalation reactions are given below for a pristine anionic clay of the general formula: Mg₂Al(OH)₆.Cl.yH₂O (note that this is equivalent to [Mg₁₋ₓAlₓ(OH)₂]0.33 Cl.yH₂O for x=0.33). The word “pristine” is generally used to refer to the native anionic clay in its original or pre-intercalated form. After intercalation, the anion of the pristine clay is replaced by a desired anionic molecule, ion or polymeric species, hereafter referred to as the functional-active (F-A) compound.

The ion exchange reaction is represented according to equation (1):
Mg₂Al(OH)₆.Cl.yH₂O+M⁺(F-A)ⁿ⁻⇄Mg₂Al(OH)₆.(F-A).yH₂O+M⁺Cl⁻ (2)
where M is a cation (usually H⁺, Na⁺ or K⁺). Note that the reaction is an equilibrium reaction and is reversible.

The direct precipitation reaction is given by equation (2):
2 MgCl₂+AlCl₃+M⁺(F-A)ⁿ⁻⇄Mg₂Al(OH)₆.(F-A).yH₂O+7 M⁺Cl⁻ (2)
where M is a cation (again, usually H⁺, Na⁺ or K⁺). Note that the reaction is an equilibrium reaction and is reversible.

The reconstruction synthesis is given by equation (3):
3.51 Mg_{0.57}Al_{0.28}O+M⁺(F-A)ⁿ⁻⇄Mg₂Al(OH)₆.(F-A).yH₂O (3)
When M⁺=H⁺, the reconstruction synthesis is unique among the three, as the reaction has no by-products.

The ion-exchange (1) and the direct precipitation (2) intercalation reactions have a salt or an acid generated as a by-product, i.e., M⁺Cl⁻ from above. In most cases, this salt must be removed by filtration or diafiltration. Further, since the reactions are equilibrium reactions, the presence of salt by-products shifts the equilibrium in the backward direct (toward the starting components), due to Le Chateliers' principle. The backward shift in the equilibrium produces un-intercalated or “free” functional-active, and thus limits the ability of the anionic clay to act as a sequestration and release agent. Functional-active may then be lost or wasted in the filtration process. The reconstruction method (3) however, has no salt by-products and does not require the removal of the by-products via filtration. The reaction still is an equilibrium process, however, and some functional-active may remain un-intercalated (or unsequestered). The preparation in which substantially all of the functional-active remains in a sequestered state, and does not require filtration is desired.

The ability and capacity of anionic clays to sequester or intercalate anionic species may depend directly upon their ion-exchange capacity. The ion-exchange capacity of an anionic clay is defined as the number of equivalents (eq.) of anion that may be exchanged in the interlayer region per mole of anionic clay, or per gram of anionic clay (specific ion-exchange capacity). For example, Mg_{0.7}Al_{0.3}(OH)₂.0.3 NO₃.0.6H₂O (molecular weight=88.51 g/mol) has a molar exchange capacity of 0.3 eq./mole, and a specific ion-exchange capacity of 3.4 milli-eq./g. The ion-exchange capacity of an anionic clay having a general composition, [M²⁺₁₋ₓM³⁺ₓ(OH)₂](x/n) Aⁿ⁻.yH₂O, is determined by the degree of substitution of M³⁺ ion (i.e., the value of x) in the formula above, and by the charge of the anion (n⁻); where n is an integer usually between 1 and 4. Thus, for n=2, the molar ion-exchange capacity is x/2. The ion-exchange capacity is always limited by the rule of charge neutrality, such that the sum of all negative and positive charges is balanced. One skilled in the art may determine the ion-exchange capacity of a particular anionic clay using the rule of charge neutrality.

In one embodiment, the invention provides an aqueous dispersion comprising an anionic clay, an intercalated functional-active compound and optionally a polymer, wherein substantially all (e.g., at least 80 percent) of the functional-active compound is intercalated and resides between the layers of the layered host material, and the stoichiometric ratio of anionic clay to functional-active compound is between about 1.2 to 7 equivalents. The stoichiometric ratio is the molar ion-exchange capacity of the clay as defined above, and thus, the invention requires that the anionic clay be present in an excess of 1.2 to 7.0 times that required (theoretically), as calculated from the molar ion-exchange capacity, to intercalate all of the functional-active.

The invention is unique with respect to prior art intercalation compositions, in that it is directed towards the intercalation of a high percentage of the total functional-active compound in an aqueous dispersion, while not requiring a very high excess of anionic clay in the dispersion. It is preferred that greater than 80 percent, and more preferably greater than 90 percent, of the functional-active is intercalated into the layered host material. The efficiency of the intercalation process, i.e., the percentage of intercalation, may vary depending upon the nature of the functional-active species, the charge on the functional-active species, and the exact nature and composition of the layered host material. Generally, functional-actives having more than one anionic functional group per molecule intercalate with greater efficiency than singly charged functional actives. One skilled in the art may determine the efficiency of the intercalation process for a given layered host and functional-active by using the methods described herein. The efficiency of the intercalation process may thus determine the precise stoichiometric ratio employed. In accordance with certain embodiments of the invention, e.g., the anionic clay may need to be present in an amount at least 2 times, or at least 3 times, or at least 4 times that required (theoretically), as calculated from the molar ion-exchange capacity, to intercalate all of the functional-active in order to reach the desired intercalation of at least 80% of the functional-active compound. The upper limit of about 7 times the theoretical stoichiometric ratio, however, distinguishes such compositions from highly intercalated compositions of the prior art.

As the amount of anionic clay in the compositions of the invention is present in at least a slight stoichiometric excess with respect amount of functional-active compound (i.e., between about 1.2 to 7 equivalents), for charge neutrality, a second anion may be used to balance the charge of the cationic (positively charged) clay layers. It is preferred that the second anion is selected from nitrate, chloride, bromide or perchlorate (ClO₄⁻). These anions are preferred because they are very easily displaced from the clay layers, and allow for the full and complete sequestration of the functional-active.

For an anionic clays of type (I), the invention provides an aqueous dispersion having an anionic clay and a functional-active with the general formula:
[M²⁺₁₋ₓM³⁺ₓ(OH)₂]^{x+}a/n (F-A)ⁿ⁻b/p (anion2)^{p-}.yH₂O;
where F-A is the functional-active and anion2 is non-functional-active secondary anion, preferably nitrate, chloride, bromide or perchlorate (ClO₄⁻), and (n)(a)+(p)(b)=x, and (p)(b)/(n)(a) is between 0.75 and 5.0.

For an anionic clay of type (II), the invention provides an aqueous dispersion having an anionic clay and a functional-active with the general formula:
[M¹⁺M³⁺₂(OH)₆]¹⁺a/n (F-A)ⁿ⁻b/p (anion2)^{p-}.yH₂O;
where F-A is the functional-active and anion2 is non-functional-active secondary anion, preferably nitrate, chloride, bromide or perchlorate (ClO₄⁻), and (n)(a)+(p)(b)=1, and (p)(b)/(n)(a) is between 0.75 and 5.0.

In a particularly preferred embodiment, the anionic clay for practice of the invention is rehydrated from a calcined product of an anionic clay, such as hydrotalcite, Mg₂Al(OH)₆.1/2CO₃.yH₂O, Zn₂Al(OH)₆.1/2CO₃.yH₂O, Mg₂Al(OH)₆.NO₃.yH₂O and Zn₂Al(OH)₆.NO₃.yH₂O. The term “calcination” refers to a heating process whereby the layered double hydroxide, or hydrotalcite, is heated to decomposition, usually at a temperature greater than about 300° C. The thermal treatment of hydrotalcite produces a calcined product according to the reaction in equation (4).
Mg_{0.7}Al_{0.3}(OH)₂0.15CO₃.yH₂O→1.15 Mg_{0.62}Al_{0.26}O (4)
The calcined product is an amorphous mixed oxide and when placed in water, even at room temperature, has a strong tendency to rehydrate and will reform a layered double hydroxide, if a suitable anion is present. This unusual property is often referred to as “reconstruction synthesis”. The reconstruction of a layered double hydroxide from a calcined hydrotalcite product with a nitrate anion is depicted in equation (5).
1.15 Mg_{0.62}Al_{0.26}O+0.3 HNO₃→Mg_{0.7}Al_{0.3}(OH)₂.0.3 NO₃.yH₂O (5)
More generally, the reconstruction of a layered double hydroxide may be accomplished according to equation (6).
1.15Mg_{0.62}Al_{0.26}O+0.3/n H⁺Anion⁻ⁿ→Mg_{0.7}Al_{0.3}(OH)₂.0.3/n Anion.yH₂O (6)
In accordance with a particular embodiment of the invention, preparation of a composition comprising particles of an anionic clay layered host material and functional-active organic compound dispersed in an aqueous medium is performed by reconstruction synthesis employing a calcined layered double hydroxide product. Such process comprises reacting between about 1.2 to 7 equivalents of a calcined product of a layered double hydroxide with a functional-active organic compound in an aqueous medium, wherein a secondary non-functional-active ion is also reacted with the calcined product, such that the calcined product is rehydrated in the presence of the functional-active organic compound and secondary ions to form particles of an anionic clay layered double hydroxide host material with molecules of the functional-active organic compound and secondary ion intercalated between layers of the layered host material particles. In such process, amorphous calcined product solid is re-dispersed in aqueous media containing a suitable functional-active compound anion, and “reconstructs” to form the layered intercalation compound of the new anion. The “new” anionic species may be a functional-active compound chosen in accordance with the invention such that it imparts a particular chemical, pharmaceutical or biological function. The reaction has the stoichiometry as indicated in Eq. (6), and generally proceeds readily at, or just above, room temperature. The reaction is quite remarkable in that in many cases it proceeds to completion and has no by-products. Calcined derivatives of layered double hydroxides are therefore preferred sources for the anionic clays employed in the invention because they provide aqueous chemical-delivery, and/or, a drug-delivery systems which do not require filtration and washing, and are simple and inexpensive to prepare.

In such reconstruction process, the calcined layered double hydroxide, hereafter referred to as c-LDH, is provided in aqueous dispersion in a stoichiometric ratio of 1.2 to 7.0 times greater than that necessary (as calculated from the molar ion-exchange capacity) to react and intercalate the functional-active. The reconstruction intercalation thus requires the addition of a secondary anion, such as nitrate, chloride, bromide or perchlorate (ClO₄⁻), for charge neutrality. The reaction may be described as below (equation 7) for anionic clays of type (I). 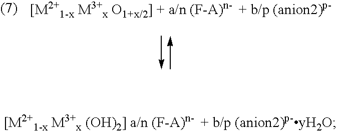
where (n)(a)+(p)(b)=x, and (p)(b)/(n)(a) is between 0.75 and 5.0. It is preferred that the anion2 is provided in the form of an acid such as HNO₃, HCl, HBr or HClO₄. It is further preferred that the pH of the reaction mixture is controlled between about 5 and 9. This is preferred because the intercalation reaction is pH dependent, and de-intercalation may occur at pH values outside of this range (absent countervailing measures).

The invention provides a composition wherein an intercalated functional-active compound which is first sequestered by the layered compound may be later released to perform a desired function. The functional active may be, e.g., a chemically-active, a biologically-active, a pharmaceutically-active, or a nutraceutically-active molecule or ion. Chemical-actives may include, e.g., photographic materials such as inhibitors, e.g., phenyl mercaptotetrazole, developers and development accelerators e.g., ascorbic acid, and other photographic materials; chemical actives may also include dyes, analytical reagents, surfactants, oxygen scavengers and antioxidants such as sulfites, and catalytic materials. Biological-actives may be chosen from, e.g., biocides, antimicrobials, antibacterials and preservatives such as benzoate or benzoic acid, sorbate or sorbic acid, ascorbate or ascorbic acid. Biological actives may also include skin, cosmetic or health care reagents. Pharmaceutically-active compounds may include, e.g., materials such as drugs, antibiotics, bio-materials such as amino-acids, peptides, proteins, therapeutics, hormones, enzymes, growth factors, and genetic materials such as RNA, DNA, and oligonucleotides. Nutraceutically-active compounds may be chosen from, e.g., vitamins, proteins, preservatives and food-additive materials. In one preferred embodiment, the functional active may be a biologically active, a pharmaceutically active, or a nutraceutically active molecule. In a further embodiment, the functional-active compound may comprise an antimicrobial compound. An antimicrobial compound is a molecule or complex that is able to inhibit the growth of micro-organisms.

In order to facilitate intercalation of a desired functional-active into an anionic clay layered host material, it may be necessary to prepare derivatives of the desired functional-active such that the compound attains a negative charge. To prepare an anionic functional-active, a carboxylic or sulfonic acid function, or a sulfate group, may be attached to the parent functional-active. It is preferred that the derivatization does not interfere with the primary function of the parent functional-active.

Compositions in accordance with various embodiments of the invention optionally may further comprise a polymer. The polymer may be employed, e.g., to further slow the rate of release of the functional active from the layered anionic clay particles. The polymer may alternatively be used to apply the composition of matter of the invention to articles such as plastic, paper, wood, glass, metal, fabric and textiles, and other materials. The polymer may be used to encapsulate the composition of matter into a pill or other ingestible tablet or capsule. It is preferred that the polymer is a biocompatible polymer. Polymer suitable for practice of the invention include polyethyleneglycol, methoxypolyethylene glycol, polypropylene glycol, dextran, polylysine, polysaccharides, polypeptides, gelatin, albumin, chitosan, cellulose, hydrogels, polyvinyl alcohol, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene, polypropylene or polyacrylonitrile. EXAMPLES

Hydrotalcite (Layered double hydroxide) was obtained from Sud Chemie having the composition Mg_{0.7}Al_{0.3}(OH)₂.0.15 CO₃. 0.6H₂O. This material was calcined before use at 500° C. for 3 h and cooled in a nitrogen atmosphere, the calcined product is hereafter referred to as c-LDH and has the approximate composition Mg_{0.62}Al_{0.26}O. Distilled water was boiled prior to use to eliminate dissolve carbon dioxide. Intercalation reactions were carried out according to the general stoichiometry of the reaction given by equation (6) above, wherein the anion is the functional-active (chemistry or drug molecule of choice), and n represents the charge on the anion. For examples in which an excess of a stoichiometric amount of c-LDH is used, 1 N nitric acid was added to adjust the pH to between about 6-8. Compounds C-1 to C-6 were employed as functional-actives.

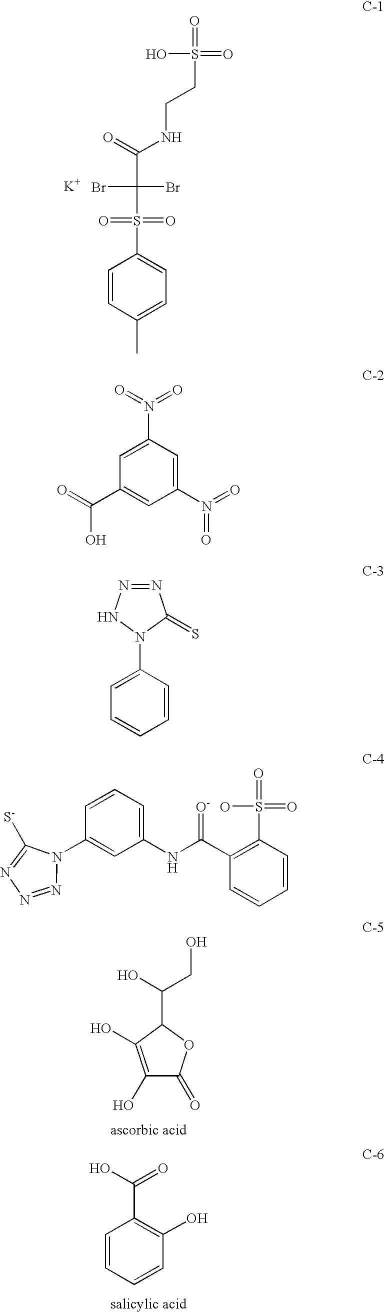

C-1 is a photographic developer inhibitor compound. C-2 is 3,5-dinitrobenzoic acid, and C-3 is phenylmercaptotetrazole, both purchased from Aldrich Chemical company. C-4 is 1-[3-(2-sulfo)benzamidophenyl]-5-mercaptotetrazole. C-5 (ascorbic acid) and C-6 (salicylic acid) were purchased from Aldrich Chemical Company.

Preparation of Aqueous dispersions. Dispersions were prepared as given below and as indicated in Table 1. After 18 h of stirring, the dispersions were centrifuged at 7500 rpm and an aliquot of the supernatant was examined by liquid chromatography-mass spectroscopy. LC-MS. The analysis give the concentration of un-intercalated guest in the supernatant. The percent uptake of the guest by the host was calculated by as follows:
% uptake=(nominal guest conc.−found conc.)/(nominal guest conc.)

The results are reported in Table 1. In each case, the nominal concentration of guest was kept between about 5.00 and 10.0 mg/ml.

### Examples 1.1-1.3

### C-LDH and C-1

### Comparison Example 1.1

0.25 g of C-1 and 1 equivalent (0.07 g) of c-LDH were combined in 50.00 ml of distilled water, and the pH adjusted to 7.0 through the addition of about 2 drops of 1 N HNO₃. The suspension was tightly sealed from the atmosphere and allowed to stir overnight at 25° C. and then analyzed as defined above; the results are given in Table 1.

### Example 1.2

0.25 g of C-1 and 2 equivalents (0.140 g) of c-LDH were combined in 50.00 ml of distilled water, and the pH adjusted to 7.0 through the addition of about 0.97 ml of 1 N HNO₃. The suspension was tightly sealed from the atmosphere and allowed to stir overnight at 25° C. and then analyzed as defined above; the results are given in Table 1.

### Example 1.3

0.25 g of C1 and 4 equivalents (0.280 g) of c-LDH were combined in 50.00 ml of distilled water, and the pH adjusted to 7.0 through the addition of about 1.93 ml of 1 N HNO₃. The suspension was tightly sealed from the atmosphere and allowed to stir overnight at 25° C. and then analyzed as defined above; the results are given in Table 1.

### Examples 2.1-2.4

### C-LDH and C-2

### Comparison Example 2.1

0.50 g of C-2 and 1 equivalent (0.34 g) of c-LDH were combined in 50.00 ml of distilled water, and the pH adjusted to 7.0 through the addition of about 1 drop of 1 N HNO₃. The suspension was tightly sealed from the atmosphere and allowed to stir overnight at 25° C. and then analyzed as defined above; the results are given in Table 1.

### Example 2.2

0.50 g of C-2 and 2 equivalents (0.69 g) of c-LDH were combined in 50.00 ml of distilled water, and the pH adjusted to 7.0 through the addition of 2.4 ml of 1 N HNO₃. The suspension was tightly sealed from the atmosphere and allowed to stir overnight at 25° C. and then analyzed as defined above; the results are given in Table 1.

### Example 2.3

0.50 g of C-2 and 3 equivalents (1.03 g) of c-LDH were combined in 50.00 ml of distilled water, and the pH adjusted to 7.0 through the addition of 4.8 ml of 1 N HNO₃. The suspension was tightly sealed from the atmosphere and allowed to stir overnight at 25° C. and then analyzed as defined above; the results are given in Table 1.

### Example 2.4

0.50 g of C-2 and 4 equivalents (1.38 g) of c-LDH were combined in 50.00 ml of distilled water, and the pH adjusted to 7.0 through the addition of 7.1 ml of 1 N HNO₃. The suspension was tightly sealed from the atmosphere and allowed to stir overnight at 25° C. and then analyzed as defined above; the results are given in Table 1.

### Examples 3.1-3.3

### C-LDH and C3

### Comparison Example 3.1

0.50 g of C-3 and 1 equivalent (0.41 g) of c-LDH were combined in 50.00 ml of distilled water, after 1 hour, the pH was about 7.2. The suspension was tightly sealed from the atmosphere and allowed to stir overnight at 25° C. and then analyzed as defined above; the results are given in Table 1.

### Example 3.2

0.50 g of C-3 and 2 equivalents (0.82 g) of c-LDH were combined in 50.00 ml of distilled water, and the pH adjusted to 7.0 through the addition of about 2.8 ml of 1 N HNO₃. The suspension Was tightly sealed from the atmosphere and allowed to stir overnight at 25° C. and then analyzed as defined above; the results are given in Table 1.

### Example 3.3

0.50 g of C-3 and 4 equivalents (1.64 g) of c-LDH were combined in 50.00 ml of distilled water, and the pH adjusted to 7.0 through the addition of about 8.4 ml of 1 N HNO₃. The suspension was tightly sealed from the atmosphere and allowed to stir overnight at 25° C. and then analyzed as defined above; the results are given in Table 1.

### Examples 4.1-4.3

### C-LDH and C-4

### Comparison Example 4.1

0.50 g of C-4 and 1 equivalent (0.32 g) of c-LDH were combined in 50.00 ml of distilled water, and the pH adjusted to 7.0 through the addition of about 2.2 ml of 1 N HNO₃. The suspension was tightly sealed from the atmosphere and allowed to stir overnight at 25° C. and then analyzed as defined above; the results are given in Table 1.

### Example 4.2

0.50 g of C-4 and 2 equivalents (0.64 g) of c-LDH were combined in 50.00 ml of distilled water, and the pH adjusted to 7.0 through the addition of about 4.4 ml of 1 N HNO₃. The suspension was tightly sealed from the atmosphere and allowed to stir overnight at 25° C. and then analyzed as defined above; the results are given in Table 1.

### Example 4.3

0.50 g of C-4 and 3 equivalents (0.97 g) of c-LDH were combined in 50.00 ml of distilled water, and the pH adjusted to 7.0 through the addition of about 6.6 ml of 1 N HNO₃. The suspension was tightly sealed from the atmosphere and allowed to stir overnight at 25° C. and then analyzed as defined above; the results are given in Table 1.

### Examples 5.1-5.3

### C-LDH and C-5 (Ascorbic Acid)

### Comparison Example 5.1

0.50 g of ascorbic acid and 1 equivalent (0.41 g) of c-LDH were combined in 50.00 ml of distilled water, and the pH adjusted to 7.0 through the addition of about 0.8 ml of 1 N HNO₃. The suspension was tightly sealed from the atmosphere and allowed to stir overnight at 25° C. and then analyzed as defined above; the results are given in Table 1.

### Example 5.2

0.50 g of ascorbic acid and 2 equivalents (0.82 g) of c-LDH were combined in 50.00 ml of distilled water, and the pH adjusted to 7.0 through the addition of about 1.3 ml of 1 N HNO₃. The suspension was tightly sealed from the atmosphere and allowed to stir overnight at 25° C. and then analyzed as defined above; the results are given in Table 1.

### Example 5.3

0.50 g of ascorbic acid and 3 equivalents (1.23 g) of c-LDH were combined in 50.00 ml of distilled water, and the pH adjusted to 7.0 through the addition of about 2.0 ml of 1 N HNO₃. The suspension was tightly sealed from the atmosphere and allowed to stir overnight at 25° C. and then analyzed as defined above; the results are given in Table 1.

TABLE 1
Percent uptake of the functional-active by c-LDH

| Example (E) or | functional- | | weight ratio | |
|---|---|---|---|---|
| Comparison | active | stoich. ratio | c-LDH:guest | % uptake |
| example (CE) | (F-A) | c-LDH:F-A | (w/w) | guest |
| CE-1.1 | C-1 | 1 | 0.3 | 75 |
| E-1.2 | ″ | 2 | 0.6 | 94 |
| E-1.3 | ″ | 4 | 1.1 | 100 |
| CE-2.1 | C-2 | 1 | 0.7 | 56 |
| E-2.2 | ″ | 2 | 1.4 | 90 |
| E-2.3 | ″ | 3 | 2.0 | 94 |
| E-2.4 | ″ | 4 | 2.8 | 98 |
| CE-3.1 | C-3 | 1 | 0.8 | 60 |
| E-3.2 | ″ | 2 | 1.6 | 88 |
| E-3.3 | ″ | 4 | 3.3 | 91 |
| CE-4.1 | C-4 | 1 | 0.6 | 61 |
| E-4.2 | ″ | 2 | 1.2 | 100 |
| E-4.3 | ″ | 3 | 2.0 | 100 |
| CE-5.1 | C-5 | 1 | 0.82 | 52 |
| E-5.2 | ″ | 2 | 1.6 | 58 |
| E-5.3 | ″ | 3 | 2.5 | 81 |

The data of Table 1 show that the ability of c-LDH to effectively sequester functional-active chemistry from aqueous solution upon rehydration is dependent upon the number of equivalents (i.e., stoichiometric ratio) of c-LDH employed in the reaction. Employing stoichiometric ratios greater than 1 equivalent results in an increase in the sequestration of the functional-active. Greater than 80-90%, and sometimes 100% of the functional-active, may be sequestered by the anionic clay of the aqueous dispersion, depending upon the particular functional-active and particular stoichiometric ratios employed. In such cases, the dispersion may be used as prepared, and may not require further treatment such as washing or filtration. The anionic clay need only be combined with an appropriate quantity of a functional-active in aqueous dispersion and allowed to stir, typically at temperatures between about 25-70° C. The pH should be adjusted to between about 5 and 9.

### Examples 6.1-6.6

### LDH and C-6 (Salicyclic Acid)

The following examples compare the efficiency of reconstruction synthesis and ion-exchange intercalation reactions.

### Comparison Example 6.1

0.50 g of C-6 (salicylic acid) and 3.8 equivalents (1.08 g) of hydrotalcite (Mg_{0.7}Al_{0.3}(OH)₂.0.15 CO₃.0.6H₂O) were combined in 50.00 ml of distilled water, and the pH adjusted to 7.0. The suspension was tightly sealed from the atmosphere and allowed to stir overnight at 50° C. and then analyzed as defined above; the results are given in Table 2.

### Comparison Example 6.2

0.50 g of salicylic acid and 7.6 equivalents (2.16 g) of hydrotalcite (Mg_{0.7}Al_{0.3}(OH)₂.0.15 CO₃.0.6H₂O) were combined in 50.00 ml of distilled water, and the pH adjusted to 7.0. The suspension was tightly sealed from the atmosphere and allowed to stir overnight at 50° C. and then analyzed as defined above; the results are given in Table 2.

### Comparison Example 6.3

0.50 g of salicylic acid and 11.3 equivalents (3.24 g) of hydrotalcite (Mg_{0.7}Al_{0.3}(OH)₂.0.15 CO₃.0.6H₂O) were combined in 50.00 ml of distilled water, and the pH adjusted to 7.0. The suspension was tightly sealed from the atmosphere and allowed to stir overnight at 50° C. and then analyzed as defined above; the results are given in Table 2.

### Example 6.4

0.50 g of salicylic acid and 2 equivalents (1.06 g) of c-LDH were combined in 50.00 ml of distilled water, and the pH adjusted to 6.7 through the addition of about 3.6 ml of 1 N HNO₃. The suspension was tightly sealed from the atmosphere and allowed to stir overnight at 25° C. and then analyzed as defined above; the results are given in Table 2.

### Example 6.5

0.50 g of salicylic acid and 3 equivalents (1.59 g) of c-LDH were combined in 50.00 ml of distilled water, and the pH adjusted to 7.1 through the addition of about 7.2 ml of 1 N HNO₃. The suspension was tightly sealed from the atmosphere and allowed to stir overnight at 25° C. and then analyzed as defined above; the results are given in Table 2.

### Example 6.6

0.50 g of salicylic acid and 4 equivalents (2.11 g) of c-LDH were combined in 50.00 ml of distilled water, and the pH adjusted to 6.9 through the addition of about 10.9 ml of 1 N HNO₃. The suspension was tightly sealed from the atmosphere and allowed to stir overnight at 25° C. and then analyzed as defined above; the results are given in Table 2.

TABLE 2
Comparison of the efficiency of the reconstruction synthesis (examples)
and the ion-exchange intercalation reactions (comparison examples).

| Example (E) or | | | |
|---|---|---|---|
| Comparison | functional- | stoich. ratio | % uptake |
| example (CE) | active (F-A) | c-LDH:F-A | guest |
| CE-6.6 | C-6 | 3.8 | 51 |
| CE-6.2 | ″ | 7.6 | 45 |
| CE-6.3 | ″ | 11.3 | 57 |
| E-6.4 | ″ | 2 | 72 |
| E-6.5 | ″ | 3 | 78 |
| E-6.6 | ″ | 4 | 83 |

The data of Table 2 indicate that the compositions prepared by the reconstruction method have a significantly improved uptake or sequestration of the functional-active compound. The degree of sequestration is poor for all comparison examples even when the stoichiometric ratio of the hydrotalcite is very high.

The invention has been described in detail with particular reference to certain preferred embodiments thereof, but it will be understood that variations and modifications can be effected within the spirit and scope of the invention.

1. A composition comprising particles of an anionic clay layered host material and functional-active organic compound dispersed in an aqueous medium, wherein at least 80% of the functional-active organic compound in the dispersion is intercalated between layers of the layered host material particles, and the stoichiometric ratio of anionic clay to functional-active compound is between about 1.2 to 7 equivalents.

2. The composition of claim 1 wherein the anionic clay layered host material comprises a layered double hydroxide intercalated with the functional-active compound and a secondary anion and has the general formula:
[M²⁺₁₋ₓM³⁺ₓ(OH)₂]^{x+}a/n (F-A)ⁿ⁻b/p (anion2)^{p-}.yH₂O;
where M²⁺ is a divalent metal selected from Ca, Mg, Mn, Co, Ni, Cu, Zn, and Cd; M³⁺ is a trivalent metal selected from Cr, Fe, Al, Ga, In, Mo; x may be any rational number greater than 0 and less than 1; F-A is the functional-active compound; anion2 is a secondary non-functional-active ion; n and p are integers; y may be any rational number between 0 and 10; and (n)(a)+(p)(b)=x, and (p)(b)/(n)(a) is between 0.75 and 5.0.

3. The composition of claim 2, wherein the anion2 is selected from nitrate, chloride, bromide, carbonate, bicarbonate or perchlorate (ClO₄⁻).

4. The composition of claim 1 wherein the anionic clay layered host material comprises a layered double hydroxide intercalated with the functional-active compound and a secondary anion and has the general formula:
[M¹⁺M³⁺₂(OH)₆]¹⁺a/n (F-A)ⁿ⁻b/p (anion2)^{p-}.yH₂O;
where M¹⁺ is a mono-valent metal selected from Li, Na, K, Rb or Cs; M³⁺ is a trivalent metal selected from Cr, Fe, Al, Ga, In, Mo; F-A is the functional-active compound; anion2 is a secondary non-functional-active ion; n and p are integers; y may be any rational number between 0 and 10; and (n)(a)+(p)(b)=1, and (p)(b)/(n)(a) is between 0.75 and 5.0.

5. The composition of claim 4 wherein the anion2 is selected from nitrate, chloride, bromide, carbonate, bicarbonate or perchlorate (ClO₄⁻).

6. The composition of claim 1, comprising anionic clay particles of average particle size less than 2 microns.

7. The composition of claim 1, comprising anionic clay particles of average particle size less than 0.2 microns.

8. The composition of claim 1, wherein the pH of the aqueous dispersion is between about 5-9.

9. The composition of claim 1, wherein the functional-active compound comprises a biologically active, a pharmaceutically active, or a nutraceutically active compound.

10. The composition of claim 1, wherein the functional-active compound comprises a biologically active compound.

11. The composition of claim 1, wherein the functional-active compound comprises a pharmaceutically active compound.

12. The composition of claim 1, wherein the functional-active compound comprises a nutraceutically active compound.

13. The composition of claim 1, wherein the functional-active compound comprises an antimicrobial compound.

14. The composition of claim 1, further comprising a polymer.

15. The composition of claim 14, wherein the polymer comprises a biocompatible polymer.

16. The composition of claim 14, wherein the polymer comprises polyethyleneglycol, methoxypolyethylene glycol, polypropylene glycol, dextran, polylysine, polysaccharides, polypeptides, gelatin, albumin, chitosan, cellulose, hydrogels, polyvinyl alcohol, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene, polypropylene or polyacrylonitrile.

17. The composition of claim 14, wherein the functional-active compound is a pharmaceutically active compound and wherein the polymer comprises a biocompatible polymer.

18. The composition of claim 17, wherein the biocompatible polymer is formed into a gel capsule and the gel capsule contains the aqueous dispersion.

19. The composition of claim 1, wherein at least 90% of the functional-active organic compound in the dispersion is intercalated between layers of the layered host material particles.

20. The composition of claim 1, wherein the stoichiometric ratio of anionic clay to functional-active compound is at least about 2 equivalents.

21. The composition of claim 1, wherein the stoichiometric ratio of anionic clay to functional-active compound is at least about 3 equivalents.

22. The composition of claim 1, wherein the stoichiometric ratio of anionic clay to functional-active compound is at least about 4 equivalents.

23. A process for the preparation of a composition comprising particles of an anionic clay layered host material and functional-active organic compound dispersed in an aqueous medium, the process comprising reacting between about 1.2 to 7 equivalents of a calcined product of a layered double hydroxide with a functional-active organic compound in an aqueous medium, wherein a secondary non-functional-active ion is also reacted with the calcined product, such that the calcined product is rehydrated in the presence of the functional-active organic compound and secondary ions to form particles of an anionic clay layered double hydroxide host material with molecules of the functional-active organic compound and secondary ion intercalated between layers of the layered host material particles.

24. The process of claim 23, wherein at least 80% of the functional-active organic compound is intercalated between layers of the layered host material particles.

25. The process of claim 23, wherein at least 90% of the functional-active organic compound is intercalated between layers of the layered host material particles.

26. The process of claim 23, wherein the stoichiometric ratio of the calcined product of a layered double hydroxide to functional-active compound is at least about 2 equivalents.

27. The process of claim 23, wherein the stoichiometric ratio of the calcined product of a layered double hydroxide to functional-active compound is at least about 3 equivalents.

28. The process of claim 23, wherein the stoichiometric ratio of the calcined product of a layered double hydroxide to functional-active compound is at least about 4 equivalents.

29. The process of claim 23, wherein the resulting intercalated anionic clay layered double hydroxide material has the general formula:
[M²⁺₁₋ₓM³⁺ₓ(OH)₂]^{x+}a/n (F-A)ⁿ⁻b/p (anion2)^{p-}.yH₂O;
where M²⁺ is a divalent metal selected from Ca, Mg, Mn, Co, Ni, Cu, Zn, and Cd; M³⁺ is a trivalent metal selected from Cr, Fe, Al, Ga, In, Mo; x may be any rational number greater than 0 and less than 1; F-A is the functional-active compound; anion2 is a secondary non-functional-active ion; n and p are integers; y may be any rational number between 0 and 10; and (n)(a)+(p)(b)=x, and (p)(b)/(n)(a) is between 0.75 and 5.0.

30. The process of claim 29, wherein the anion2 is selected from nitrate, chloride, bromide, carbonate, bicarbonate or perchlorate (ClO₄⁻).

31. The process of claim 23, wherein the resulting intercalated anionic clay layered double hydroxide material has the general formula:
[M¹⁺M³⁺₂(OH)₆]¹⁺a/n (F-A)ⁿ⁻b/p (anion2)^{p-}.yH₂O;
where M¹⁺ is a mono-valent metal selected from Li, Na, K, Rb or Cs; M³⁺ is a trivalent metal selected from Cr, Fe, Al, Ga, In, Mo; F-A is the functional-active compound; anion2 is a secondary non-functional-active ion; n and p are integers; y may be any rational number between 0 and 10; and (n)(a)+(p)(b)=1, and (p)(b)/(n)(a) is between 0.75 and 5.0.

32. The process of claim 31 wherein the anion2 is selected from nitrate, chloride, bromide, carbonate, bicarbonate or perchlorate (ClO₄⁻).

33. The process of claim 23, wherein the pH of the aqueous dispersion is maintained between about 5-9.
